SEQUENCE LISTING (5 sequences)

SEQ ID NO: 1 - DNA, 1292 bp, Homo sapiens
  CDS (181)..(1200)
gttaactgcatttgcagctagaagttaggctctgattcactgttttgtattttctaaaagggttatatgtaatttgaaagatagacctgccaagacgtgagatctgtgttctccttggttagagctaacatttttggtgaggaaagcactgcaggagcaggctggcacagagaagaggacatgcggaaggagctccagctctccctgtcagtcaccttgctgcttgtctgtggcttcctctaccagttcaccctgaagtccagctgcctcttctgtttgccttctttcaagtcccaccaggggctggaagccctcctgagccacagacgtggcattgtgtttctagagacctcagagagaatggagccaccccatttggtctcctgttccgtagagtctgctgccaagatttatcctgagtggcctgtggtgttctttatgaagggtcttactgattccacaccgatgccctcaaactccacatacccagctttttccttcctgtcagcaatagacaacgttttcctcttccctttggatatgaaaaggctgcttgaagacacaccattgttttcatggtacaatcaaatcaacgccagcgcagagagaaactggctccacatcagctcggatgcatcccgcctggccatcatctggaaatacggtggcatctacatggacaccgatgtcatctccatcaggcccatccctgaggagaactttttggctgcgcaggcttctcggtactctagtaatggaatatttgggttcctcccccaccacccctttttgtgggaatgcatggaaaactttgttgaacactataattcagccatttggggcaaccaaggccctgagttgatgacaaggatgttgagggtatggtgtaaacttgaagacttccaggaggtgagcgacctcaggtgtctgaacatatccttcttacacccccaaagattttaccccatctcctatcgagagtggaggcgctactatgaagtgtgggatacagagccaagcttcaatgtctcttatgccctgcatttgtggaaccacatgaaccaggaggggcgggctgtgattagaggaagcaacacactggtggaaaatctctatcgcaagcactgtcccaggacttacagggacctgattaaaggcccagaggggtcagtgactggggagctgggtccaggtaacaaataaagctaacactcgtttgctgctgctgcagtgtggaaatggaaaaaaaaaaaaaaaaaaaaaaaaaaaaaaaaaaaaaaaaaaaaaaaaaa

SEQ ID NO: 2 - protein, 340 aa, Homo sapiens
MRKELQLSLSVTLLLVCGFLYQFTLKSSCLFCLPSFKSHQGLEALLSHRRGIVFLETSERMEPPHLVSCSVESAAKIYPEWPVVFFMKGLTDSTPMPSNSTYPAFSFLSAIDNVFLFPLDMKRLLEDTPLFSWYNQINASAERNWLHISSDASRLAIIWKYGGIYMDTDVISIRPIPEENFLAAQASRYSSNGIFGFLPHHPFLWECMENFVEHYNSAIWGNQGPELMTRMLRVWCKLEDFQEVSDLRCLNISFLHPQRFYPISYREWRRYYEVWDTEPSFNVSYALHLWNHMNQEGRAVIRGSNTLVENLYRKHCPRTYRDLIKGPEGSVTGELGPGNK

SEQ ID NO: 3 - DNA, 25 bp, Artificial
  5′ primer for RT-PCR
gttttcctcttccctttggatatga

SEQ ID NO: 4 - DNA, 22 bp, Artificial
  3′ primer for RT-PCR
agctgatgtggagccagtttct

SEQ ID NO: 5 - DNA, 26 bp, Artificial
  Probe for Quantative-PCR
tggtacaatcaaatcaacgccagcgc

(19) United States
(12) Patent Application Publication
(10) Pub. No.: US 2006/0216715 A1
(43) Pub. Date: Sep. 28, 2006

(54) Method of detecting cancer

(76) Inventors: Jun Nakayama, Matsumoto-shi (JP); Satoshi Ishizone, Matsumoto-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/563,540

(22) PCT Filed: Jun. 28, 2004
(86) PCT No.: PCT/JP04/09126

§ 371 Date: Dec. 22, 2005

(51) Int. Cl. C12Q 1/68; C12Q 1/48

(52) U.S. Cl. 435/6; 435/15

(57) ABSTRACT

Cancer is detected by measuring the expression level of α 1,4-N-acetyl-D-glucosamine transferase gene in a body fluid collected from a living body to correlate the measurement value with presence or absence, development, degree of progress, or prognosis of cancer, wherein expression level of said gene is measured by detecting an arbitrary region consisting of continuous nucleotides having a length of 70 to 139 bp in the nucleotide sequence of SEQ ID NO:1.

### TECHNICAL FIELD

The resent invention relates to a method of detecting cancer by measuring expression level of a particular region of a gene encoding a glycosyltransferase to correlate the measurement value with the presence or absence, development, degree of progress, or prognosis of cancer; and to a kit for detecting cancer by performing the method.

### BACKGROUND ART

In the present specification, N-acetyl-D-glucosamine is referred to as “GlcNAc”. Meanwhile, sugars and sugar residues are regarded as D-forms unless otherwise specified.

Conventionally, various tumor markers or the like have been used as an indicator for detecting cancer, but their sensitivities were not always sufficient. Therefore, in addition to so-called tumor markers, there have been made attempts to correlate a variation in gene expression will cancer detection. JP 200146077 A and Lab. Invest., Vol. 83, No. 2 (2003), 187-197 disclose an enzyme that transfers GlcNAc to a mucin-type sugar chain by an α-1,4 linkage and DNA thereof, and also disclose a technique for applying the gene to a method of detecting cancer based on different expression level of said gene in stomach cancer or pancreatic cancer. However, the sensitivity of such a technique was insufficient and requires further improvement for practical use.

### DISCLOSURE OF THE INVENTION

For solving the above-described problems, the inventors of the present invention have made extensive studies, and have found that sensitivity of detecting cancer is significantly increased compared with the conventional methods when expression level of the same gene as disclosed in the above-described prior art used for detecting a particular cancer is measured by detecting a narrower region than that disclosed in the prior art, thereby the present invention has been completed.

That is, the present invention is as follows.

(1) A method of detecting cancer comprising measuring the expression level of a 1,4-N-acetyl-D-glucosamine transferase gene in a body fluid collected from a living body to correlate the measurement value with the presence or absence, development, degree of progress, or prognosis of cancer, wherein expression level of said gene is measured by detecting an arbitrary region consisting of continuous nucleotides having a length of 70 to 139 bp in the nucleotide sequence of SEQ ID NO: 1.

(2) The method of detecting cancer according to (1), wherein said region is a region consisting of a nucleotide sequence of nucleotide numbers from 520 to 628 of SEQ ID NO: 1.

(3) The method of detecting cancer according to (1) or (2), wherein said body fluid is blood or lymph.

(4) The method of detecting cancer according to any one of (1) to (3), wherein said cancer is one or more cancers selected from the group consisting of salivary gland cancer, esophageal cancer, stomach cancer, pancreatic cancer, gallbladder cancer, small intestine cancer, colon cancer, and rectal cancer.

(5) The method of detecting cancer according to any one of (1) to (3), wherein said cancer is pancreatic cancer.

(6) A method of determining a degree of progress of pancreatic cancer comprising measuring the expression level of α 1,4-N-acetyl-D-glucosamine transferase gene in a body fluid collected from a living body to correlate the measurement value with the degree of progress of the pancreatic cancer, wherein the expression level of said gene is measured by detecting an arbitrary region consisting of continuous nucleotide sequence having a length of 70 to 139 bp the nucleotide sequence of SEQ ID NO: 1.

(7) The method of determining a degree of progress of pancreatic cancer according to (6), wherein said region is a region consisting of a nucleotide sequence of nucleotide numbers from 520 to 62 8 in SEQ ID NO: 1.

(8) The method of determining a degree of progress of pancreatic cancer according to (6) or (7), wherein said body fluid is blood or lymph.

(9) A kit for detecting cancer, comprising primers for amplifying an arbitrary region consisting of continuous nucleotide sequence having a length of 70 to 139 bp in the nucleotide sequence of SEQ ID NO: 1.

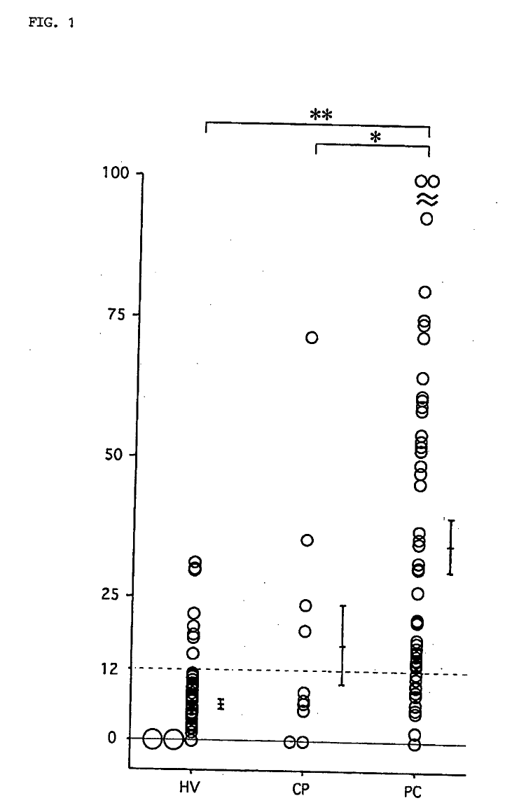

### BRIEF DESCRIPTION OF THE DRAWINGS

[FIG. 1] FIG. 1 shows distribution of the α4GnT gene expression levels (vertical axis) in healthy volunteers (HV), chronic pancreatitis patients (CP), and pancreatic cancer patients (PC). The asterisks show combinations where significant differences were observed. The symbols “*” and “**” represent P=0.015 and P=0.0046, respectively.

### DESCRIPTION OF THE PREFERRED EMBODIMENTS

Hereinafter, the present invention will be described in more detail by way of embodiments.

1. Detection Method of the Present Invention

The detection method of the present invention is a method of detecting cancer by measuring the expression level of an α 1,4-N-acetyl-D-glucosamine transferase gene in a body fluid collected from a living body to correlate the measurement value with the presence or absence, development, degree of progress, or prognosis of cancer, wherein the expression level of the gene is measured by detecting an arbitrary region consisting of continuous nucleotide sequence having a length of 70 to 139 bp in the nucleotide sequence of SEQ ID NO: 1.

The “body fluid” in the detection method of the present invention is preferably saliva, blood, lymph, gastric juice, pancreatic juice, and intestinal fluid, more preferably blood and lymph, and particularly preferably blood. When blood is used as the “body fluid”, it is preferred that quantification is performed using cDNA prepared as follows: a cell fraction containing nucleus is fractionated from blood, and total RNA is extracted from the fraction, followed by preparation of cDNA using a reverse transcriptase or the like in accordance with the conventional method.

An example of the α 1,4-N-acetyl-D-glucosamine transferase (hereinafter also referred to as “α4GnT”) gene includes a gene having the nucleotide sequence of SEQ ID NO: 1. Meanwhile, it is expected that nucleotide substitution may occur in the gene depending on a race, so that the α4GnT gene may be a gene that hybridizes with a polynucleotide having the nucleotide sequence of SEQ ID NO: 1 under stringent conditions as long as the gene encodes a protein having an α4GnT-transferring activity. Examples of the stringent conditions include conditions comprising performing washing once, more preferably two or three times at 60° C. at a salt concentration corresponding to 1×SSC, 0.1% SDS, preferably 0.1×SSC, 0.1% SDS.

In the detection method of the present invention, measurement of the expression level of the α4GnT gene is performed by detecting an arbitrary region consisting of preferably continuous nucleotides having a length of 70 to 139 bp, more preferably continuous nucleotides having a length of 80 to 129 bp, further more preferably continuous nucleotides having a length of 90 to 119 bp, particularly preferably continuous nucleotides having a length of 100 to 108 bp in the nucleotide sequence of SEQ ID NO: 1. Examples of the above-described region to be used-for the detection method of the present invention include a region consisting of a nucleotide sequence ranging from the 520th nucleotide to 628th nucleotide in SEQ ID NO: 1.

The “measurement of the expression level of the α4GnT gene” in the detection method of the present invention means measurement of the expression level of the α4GnT gene in a cell that is present in a body fluid, that is, measurement of the level of mRNA of α4GnT. Specific examples of the measurement method include: a method of detecting the above-described region after synthesizing cDNA from mRNA or total RNA extracted from a body fluid in accordance with the conventional method, by amplifying the region from the cDNA by the polymerase chain reaction method (PCR method) or the like; or by means of a DNA chip or the like using a probe corresponding to the above-described region. Of these, the PCR method is preferable, and the quantitative PCR method using a fluorescent probe may be used. However, the method of the present invention is not limited thereto, and other methods can be used as long as they enable measurement (quantification) of the expression level of the above-described gene.

Cancer can be easily detected by correlating the expression level of the α4GnT gene in a body fluid collected from a living body, which is measured using one of the above-described methods, with the presence or absence, development, degree of progress, or prognosis of cancer.

“To correlate the measurement value with the presence or absence, development, degree of progress, or prognosis of cancer” in the detection method of the present invention means that the expression level of the α4GnT gene is used as a qualitative or quantitative indicator of cancer indicating the presence or absence, presence or absence of metastasis, degree of progress, or degree of healing.

It also means by the above-mentioned correlation a state where a measurement value has varied compared with that in a healthy state, and the state can be determined as, for example, “cancer is present, cancer is developing, cancer is progressing to Stage II or more, or cancer is developing/regressing and therefore prognosis is bad/good”.

Herein the “measurement value” may be an absolute amount of the number of DNA copies in the PCR method, amount of hybridized DNA in the hybridization method, or the like but is not limited thereto. For example, by setting an internal standard, and the ratio to the internal standard may be used as the measurement value. A glyceraldehyde-3-phosphate dehydrogenas (hereinafter also referred to as “GAPDH”) gene which is generally used can be used as an internal standard.

The “variation” in the detection method of the present invention is preferably an “increase” in the measurement value. The ratio of the expression level of the α4GnT gene to that of the GAPDH gene in a healthy volunteer (the measurement value of the α4GnT gene/the measurement value of the GAPDH gene) is less than 12×10⁻⁷, so that in the case that the value is higher than such a relative value (12×10⁻⁷) (critical value), a person may be judged as “cancer is present”. If necessary, such a critical value can be adjusted depending on detection sensitivity or the like. Meanwhile, for example, when samples collected from a patient at different time points are compared, the patient may be judged as: “cancer is developing (progressing)” if the above-described relative value becomes larger; “cancer is getting better” if the value becomes smaller; “prognosis is bad or good”; or the like.

The “cancer” to be detected by the detection method of the present invention preferably is cancer of a digestive organ or an associated organ thereof, and is particularly preferably any one of salivary gland cancer, esophageal cancer, stomach cancer, pancreatic cancer, gallbladder cancer, small intestine cancer, colon cancer, and rectal cancer. Of those, stomach cancer, pancreatic cancer, small intestine cancer, or colon cancer is preferable; stomach cancer or pancreatic cancer is more preferable; and pancreatic cancer is particularly preferable.

Meanwhile, a cancer marker that has conventionally been used for detecting cancer such as a carcinoembryonic antigen (hereinafter also referred to as “CEA”) or sialyl Lewis A (hereinafter also referred to as ”CA19-9”) is not sufficient to detect early cancer (before Stage II). Therefore, early cancer has been detected by means of such a cancer marker in combination with measuring serum elastase.

On the other hand, it is clearly understood that the detection method of the present invention has extremely excellent detection sensitivity even in Stage II cancer, and early cancer can be detected only by performing the detection method of the present invention.

This has revealed that the detection method of the present invention is extremely useful for detecting early cancer (Stage II), therefore the method may be used as a method of detecting early cancer.

Moreover, it was found in the measurement by the detection method of the present invention that the measurement values vary according to cancer progression periods of Stage II, Stage III, and Stage IV, particularly in a pancreatic cancer patient, and it was also found that the detection method of the present invention can be used for determining the progress degree of pancreatic cancer based on the variation. That is, for example, when the expression levels of the α4GnT gene and the GAPDH gene (as an internal standard gene) are measured using a body fluid, particularly preferably peripheral blood, to calculate a value by multiplying the ratio thereof by 10⁷, the degree can be defined as Stage IV if the measurement value is 35 or more, the degree can be defined as Stage III if the measurement value is 15 to 35, and the degree can be defined as Stage II if the measurement value is 13 to 15. The ranges of the numerical values (critical values) of the ratio can be appropriately adjusted if necessary.

In general, it is known that differential diagnosis between pancreatitis and pancreatic cancer is clinically difficult. However, when the detection method of the present invention is used, the measurement value in a pancreatitis patient is significantly different from that in a pancreatic cancer (cancer) patient, so that the method can be used to distinguish pancreatitis from pancreatic cancer.

2. Kit of the Present Invention

The kit of the present invention is a kit for detecting cancer, which contains primers for amplifying an arbitrary nucleic acid (polynucleotide) having continuous nucleotide sequence having a length of 70 to 139 bp in the nucleotide sequence of SEQ ID NO: 1. The length of each primer is not particularly limited as long as the primer can amplify the above-described nucleic acid, but for example, each primer consists of preferably 20 to 26 nucleotides, more preferably 22 to 25 nucleotides.

The kit of the present invention is a kit for performing the detection method of the present invention.

The “nucleic acid having a nucleotide sequence having a length of 70 to 139 bp” in the kit of the present invention is not particularly limited as long as it is an nucleic acid having continuous nucleotide sequence having a length of 70 to 139 bp that is a part of the nucleic acid of SEQ ID NO: 1, but particularly preferable is a nucleic acid-consisting of a nucleotide sequence ranging from the 520th nucleotide to 628th nucleotide in SEQ ID NO: 1.

The “primers” in the kit of the present invention are not particularly limited as long as they can at amplify the above-described “nucleotide sequence having a length of 70 to 139 bp”, but examples of primers for amplifying the above-described “nucleic acid consisting of a nucleotide sequence ranging from the 520th nucleotide to 628th nucleotide in SEQ ID NO: 1”, which is one of preferable examples of the nucleic acid, include a 5′-primer of SEQ ID NO: 3 and a 3′-primer of SEQ ID NO: 4.

In addition to the above-described “primers”, the kit of the present invention may further contains: a probe for detecting amplified products obtained by amplification using the primers (for example, DNA having the nucleotide sequence of SEQ ID NO: 5); reagents such as a reverse transcriptase and a DNA polymerase; software for displaying the results of disease detection by inputting measurement values; and the like.

### EXAMPLES

Hereinafter, the present invention will be described more specifically by examples.

### Example 1

From each of 55 patients suffering from pancreatic cancer who had given informed consent (patients who had been diagnosed as pancreatic cancer by comprehensive diagnosis), 10 chronic pancreatitis patients (patients who had been diagnosed as chronic pancreatitis by comprehensive diagnosis), and 70 healthy volunteers, 5 ml of peripheral blood was collected, and a cell fraction containing nucleus was fractioned from the blood. Then, total RNA was extracted in accordance with a conventional method, and 2U of DNaseI (manufactured by Ambion Inc.) was added thereto. Thereafter, 200U of a reverse transcriptase (manufactured by Invitrogen Corporation) was added thereto, and incubated for 55 minutes to synthesize cDNA.

The quantitative-PCR method was performed using the resultant cDNA, the 5′-primer of SEQ ID NO: 3, the 3′-primer of SEQ ID NO: 4, and a probe of SEQ ID NO: 5 (TaqMan probe: to which a fluorescent dye (5′-FAM) and a quencher (3′-TAMURA) are conjugated (manufactured by Applied Biosystems Japan Ltd.)) by ABI PRISM 7700 (manufactured by Applied Biosystems Japan Ltd.).

Quantification was performed by the multiplex PCR method in which measurement is performed by amplifying a part of the α4GnT gene using the above-described primers and probe, as well as a cDNA of glyceraldehyde-3-phosphate dehydrogenase (hereinafter also referred to as “GAPDH”) as an internal standard gene, and a numerical value calculated by multiplying “the copy number of amplified products of α4GnT/the copy number of GAPDH” by 10⁷ was defined as the “expression level of α4GnT” (hereinafter simply referred to as “expression level”).

A receiver operating characteristics curve was created by using the above-described expression levels, and it was found that the group of pancreatic cancer patient and the group of healthy volunteer were clearly differentiated by a cut-off value of 12. Thus, analysis was performed by classifying the persons with the value of 12 or less as a healthy group, and the persons with the value of more than 12 as a group suspected of having pancreatic cancer.

As a result, the positive ratio among the entire pancreatic cancer patient group was found to be 76.4, while the expression level was found to be 35.7±4.9. In addition, the numbers of pancreatic cancer-positive patients in each disease stage were found to be 0/1 patient (0%) fo:r Stage 0 (a state where cancer cells are localized in the epithelium (intraepitherlia carcinoma)), 2/3 patients (66.7%) for Stage II, 6/8 patients (75.0%) for Stage III, and: 34/43 patients (79.1%) for Stage IV, while the expression levels were found to be 24.8±12.5 for Stage II, 29.9±9.2 for Stage III, and 38.3±5.9 for Stage IV and were apt to increase with progression of the disease stage (Table 1). Meanwhile, CEA and CA19-9 in serum were also measured at the same time, and the positive ratios among the entire pancreatic cancer patient group were found to be 44.4% and 76.4%, respectively. However, the patients with Stage II pancreatic cancer were judged as negative by the method with both CEA and CA19-9 (Table 1). It was found that the detection method of the present invention enables earlier detection of pancreatic cancer than the methods of detecting pancreatic cancer using the conventional markers for pancreatic cancer.

TABLE 1

| | Expression | CEA | CA19-9 |
|---|---|---|---|
| Stage | level | (>2.5 ng · ml) | (>37 U/ml) |
| 0 | 0/1 (0%) | 0/1 (0%) | 0/1 (0%) |
| II | 2/3 (66.7%) | 0/3 (0%) | 0/3 (0%) |
| III | 6/8 (75.0%) | 3/8 (37.5%) | 6/8 (75.0%) |
| IV | 34/43 (79.1%) | 21/42 (50.0%) | 34/42 (80.9%) |
| Total | 42/55 (76.4%) | 24/54 (44.4%) | 40/54 (74.6%) |

Meanwhile, a study was performed on tumors from different occupation sites in pancreas, and there was no significant difference between the group of cancer of head of pancreas and the group of cancer of tail of pancreas. In addition, by a histopathologic study for 24 cases of resectable pancreatic cancer, there were no significant differences among the expression level and presence or absence of vascular invasion, presence or absence of lymph node metastasis, and a degree of differentiation of a cancer cell.

The expression level in the healthy volunteers was found to be 7.2±0.9, and the value was significantly lower than that in the pancreatic cancer patients (Bonferroni test, P=0.0046). Meanwhile, the expression level in the chronic pancreatitis patient group was found to be 17.8±6.9, and the value was significantly lower than that in the pancreatic cancer patient group (Bonferroni test, P=0.015) (FIG. 1). Therefore, it was found that the detection method of the present invention enables detection of pancreatic cancer as well as differential diagnosis between pancreatic cancer and chronic pancreatitis.

### INDUSTRIAL APPLICABILITY

The present invention provides a novel method of detecting cancer, a method of detecting degree of progress of pancreatic cancer, and a kit of detecting cancer.

1. A method of detecting cancer comprising measuring the expression level of α-1,4-N-acetyl-D-glucosamine transferase gene in a body fluid collected from a living body to correlate the measurement value with the presence or absence, development, degree of progress, or prognosis of cancer, wherein expression level of said gene is measured by detecting an arbitrary region consisting of continuous nucleotides having a length of 70 to 139 bp in the nucleotide sequence of SEQ ID NO: 1.

2. The method of detecting cancer according to claim 1, wherein said region is a region consisting of a nucleotide sequence of nucleotide numbers from 520 to 628 of SEQ ID NO: 1.

3. The method of detecting cancer according to claim 1, wherein said body fluid is blood or lymph.

4. The method of detecting cancer according to claim 1, wherein said cancer is one or more cancers selected from the group consisting of salivary gland cancer, esophageal cancer, stomach cancer, pancreatic cancer, gallbladder cancer, small intestine cancer, colon cancer, and rectal cancer.

5. The method of detecting cancer according to claim 1, wherein said cancer is pancreatic cancer.

6. A method of determining a degree of progress of pancreatic cancer comprising measuring the expression level of α-1,4-N-acetyl-D-glucosamine transferase gene in a body fluid collected from a living body to correlate the measurement value with the degree of progress of the pancreatic cancer, wherein the expression level of said gene is measured by detecting an arbitrary region consisting of continuous nucleotide sequence having a length of 70 to 139 bp in the nucleotide sequence of SEQ ID NO: 1.

7. The method of determining a degree of progress of pancreatic cancer according to claim 6, wherein said region is a region consisting of a nucleotide sequence of nucleotide numbers from 520 to 628 in SEQ ID NO: 1.

8. The method of determining a degree of progress of pancreatic cancer according to claim 6, wherein said body fluid is blood or lymph.

9. A kit for detecting cancer, comprising primers for amplifying an arbitrary region consisting of continuous nucleotide sequence having a length of 70 to 139 bp in the nucleotide sequence of SEQ ID NO: 1.
